(19) United States
(12) Patent Application Publication
(10) Pub. No.: US 2026/0315345 A1
(43) Pub. Date: Oct. 15, 2026

(54) METHOD FOR ENHANCING FRAGRANCE SOLUBILITY AND POTENCY USING SUPERCRITICAL GAS SATURATION IN A PRESSURE REACTOR

(71) Applicant: Robert Falken, San Marcos, CA (US)

(72) Inventor: Robert Falken, San Marcos, CA (US)

(21) Appl. No.: 19/174,920

(22) Filed: Apr. 10, 2025

(51) Int. Cl. A61K 8/34; A61Q 13/00; B01J 3/00

(52) U.S. Cl. CPC A61K 8/34; B01J 3/008; A61Q 13/00

(57) ABSTRACT

The present invention relates to a method for enhancing solubility, potency, and longevity of a fragrance composition. In exemplary embodiments, the method comprises: preparing an initial fragrance solution of ethanol (10%-99% by volume), one or more fragrance ingredients, and optionally propylene glycol (0.1%-10%); saturating the initial fragrance solution in a pressure reactor (at a volume of 50 mL-1,000 L) with supercritical gas (e.g., CO2) at 50-500 bar and 31° C.-60° C. for 1-168 hours; depressurizing the pressure reactor to ambient pressure; and recovering an enhanced fragrance solution. In one variant, raw fragrance plant materials can optionally be used to integrate aromatics into the initial fragrance solution. The recovered enhanced fragrance solution exhibits enhanced solubility (measurable by GC-MS), potency, and longevity of the one or more fragrance ingredients compared to a fragrance solution control sample (two weeks+ambient aging), which is scalable for perfumery.

### PRIORITY

This application claims priority to co-owned and co-pending U.S. Provisional Application Ser. No. 63/691,953, filed on Sep. 6, 2023, and entitled “Method for Enhancing Fragrance Solubility and Potency Using Supercritical Gas Saturation in Autoclave or High-Pressure Reactor,” which is incorporated into the present application by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records but otherwise reserves all copyright rights whatsoever.

### BACKGROUND OF THE INVENTION

The fragrance industry continuously seeks innovative methods to enhance the solubility, potency, and longevity of fragrance compositions, such as perfumes and scented products. Traditional fragrance formulation methods, such as extraction (e.g., steam distillation or solvent extraction), focus on isolating essential oils from natural materials. These methods often face limitations in solubility, efficiency, and scalability, particularly for polar fragrance ingredients, prompting the need for advanced techniques to improve fragrance solutions.

Conventional perfumery involves maceration—extracting aromatic compounds from raw materials via diffusion and solubility—and maturation, a prolonged aging process (e.g., two weeks or more) wherein reactions like oxidation and esterification refine the fragrance, enhancing complexity and stability. The present invention, producing enhanced fragrance solutions through supercritical gas saturation, addresses the extended timelines and inefficiencies of traditional maturation.

Building on this, traditional maturation relies on a prolonged process to achieve a balanced and potent fragrance composition. After blending with ethanol and additives, the solution is stored in cool, dark conditions within containers or maturation tanks for two weeks or more—often several months for premium fragrances—allowing components to integrate and develop fully. This approach, which may involve periodic stirring and olfactory monitoring, ensures stability and complexity but demands significant time, with higher-potency fragrances requiring extended periods due to slow molecular integration under ambient conditions. Final filtration removes impurities but remains inefficient due to prolonged processing times, environmental factors such as temperature and light, and limited scalability from its batch nature. Unlike traditional maturation, the present invention employs a rapid supercritical gas saturation process to enhance the solubility, potency, and longevity of enhanced fragrance solutions in hours to days, addressing these limitations.

In traditional perfumery, liquid ethanol, the dominant solvent, provides functional solubility for fragrance ingredients but introduces an olfactory drawback due to volatile residues from its production. Originating from raw materials (e.g., fruits, crops) via fermentation, distillation, or oxidation, these residues impart a distinctive odor that persists despite neutralization efforts, due to boiling points near ethanol's or low olfactory detection thresholds. This interference with the fragrance composition poses a challenge during traditional maturation, requiring prolonged blending over weeks or months to mitigate, or acceptance as an inherent limitation.

While traditional maturation over weeks to months blends fragrance ingredients with ethanol's residual odors, this invention's supercritical gas saturation process enhances fragrance solutions in hours to days. Operating at 50-300 bar and 31° C. to 60° C., the supercritical gas (e.g., CO2) may reduce volatile residues from liquid ethanol during saturation, removing them upon depressurization, while amplifying the solubility, potency, and longevity of the fragrance composition to mask any remaining odors. This dual mechanism-residue reduction and enhanced fragrance properties-positions the method to address ethanol's olfactory limitations efficiently, improving overall quality.

The present invention utilizes a pressure reactor—also known as a high-pressure reactor, autoclave, or pressure vessel—to facilitate the supercritical gas saturation process for enhancing fragrance solutions. This specialized equipment enables precise control over elevated, supercritical, and near-supercritical pressures and temperatures, configured to process volumes from 50 mL to 1,000 L at pressures of 50 bar to 300 bar and temperatures of 31° C. to 60° C. It distinguishes itself from the ambient-condition vessels used in traditional maturation, providing a foundation for the rapid enhancement of solubility, potency, and longevity of fragrance compositions.

By way of example, bench-scale pressure reactors such as the Parr Series 4520, available in one- and two-liter capacities, exemplify equipment capable of supporting the supercritical gas saturation process for enhancing fragrance solutions at smaller scales. Constructed from Stainless Steel and rated for pressures up to 1900 psig (approximately 131 bar) at temperatures up to 350° C. with PTFE flat gaskets, such reactors provide the durability and precise control needed for experimental development of fragrance compositions. This bench-scale foundation complements larger systems, such as the 500-liter Büchi Pilotclave, designed for commercial production within the process's scalable range of 50 mL to 1,000 L under similar supercritical conditions.

As global warming intensifies, industries worldwide aim to reduce reliance on fossil fuels and minimize carbon footprints, raising considerations about the environmental impact of supercritical gas saturation processes. The source of gases used in these applications, such as carbon dioxide (CO2) or nitrogen (N2), is critical to their sustainability. Globally, CO2 originates from diverse sources, including power plants, gasoline-powered vehicles, and industrial facilities, often containing impurities unsuitable for human-use applications like fragrance production. In contrast, processes such as fertilizer production and alcohol fermentation yield CO2 as a clean, pure by-product, while N2 can be sourced from air separation, both safe for such uses. These industries employ capture systems, enabling the storage and redistribution of high-quality CO2 and N2 to sectors like beverage manufacturing, medical applications, and supercritical gas processes, including saturation for enhancing fragrance solutions.

This invention leverages these advancements by utilizing clean, captured gases, such as CO2 from manufacturing facilities or N2 from air separation, for its supercritical gas saturation process. In a preferred embodiment, the method employs recycled CO2—sourced from processes like alcohol fermentation or fertilizer production—to enhance the solubility, potency, and longevity of initial fragrance solutions within a pressure reactor. The process may recycle the supercritical gas (e.g., CO2 or N2) within its saturation cycle, extending gas utility and reducing external demand. This approach supports a sustainable operation, aligning with environmental goals while producing safe, high-quality fragrance compositions. By integrating clean, captured gases, the invention enhances efficiency and scalability, reducing the environmental footprint compared to traditional maturation methods that lack such considerations.

Fragrance compositions, such as the initial fragrance solution, traditionally blend fragrance ingredients categorized by volatility into top, middle, and base notes, each contributing distinct olfactory characteristics. Top notes, highly volatile fragrance ingredients such as citrus or green scents (e.g., 0.1%-40% by volume), provide an initial, fleeting impression post-application but fade rapidly. Middle notes, with moderate volatility (e.g., 0.1%-60% by volume), such as floral or spicy profiles, emerge minutes later and persist for hours, forming the fragrance's core. Base notes, low-volatility fragrance ingredients like woody or musky scents (e.g., exceeding 30% by volume), dominate after several hours during dry-down, anchoring the longevity of the fragrance composition. This traditional classification, while subjective and lacking universal standards, shapes the temporal evolution of a fragrance under conventional maturation. Alternatively, fragrance ingredients may be classified as low, moderate, or high volatility based on their vapor pressure, offering a more objective assessment. Tools like Advanced Chemistry Development (ACD/Labs) software can predict vapor pressures from structural formulas, reported in Torr (1 Torr=0.133 kPa), treating natural oils, extracts, or absolutes as mixtures. Both the note-based and vapor pressure-based classifications provide valuable perspectives for formulating initial fragrance solutions, which this invention enhances through supercritical gas saturation to improve solubility, potency, and longevity across all volatility ranges.

The prolonged maturation process, though effective for integrating fragrance ingredients across volatility ranges, poses challenges in balancing their expression over time. Base notes, often relied upon at higher levels to sustain the longevity of the fragrance composition after application, can overpower top and middle notes as they fade, leading to repetitive or heavy dry-downs (e.g., dominated by materials like ethylene brassylate or Galaxolide®). Reducing base notes compromises the fragrance's longevity post-application. Moreover, the slow diffusion of traditional maturation—spanning weeks to months—limits efficiency and scalability. This trade-off underscores the need for a rapid method to integrate all fragrance ingredients, enhancing the solubility, potency, and longevity of initial fragrance solutions without sacrificing duration or over-relying on base-heavy profiles.

Traditional perfumery employs volatile and non-volatile solvents to dissolve and stabilize fragrance ingredients, influencing their integration during maturation. Volatile solvents, such as liquid ethanol in amounts of 10% to 99% by volume, are staples in fine fragrances, valued for rapid evaporation that releases top notes swiftly post-application, though their effect diminishes over time. Non-volatile solvents, such as benzyl benzoate, diethyl phthalate, and propylene glycol (e.g., 0.1% to 10% by volume), often accompany initial fragrance solutions, enhancing stability and viscosity but excluded from vapor pressure calculations due to their persistence. These solvents, whether combined with fragrance ingredients or added separately, shape the fragrance composition yet require weeks to months of traditional maturation for integration, limiting production efficiency.

In this invention, liquid ethanol (10%-99% by volume) serves as the primary volatile solvent, optionally augmented by co-solvents such as propylene glycol (0.1% to 10% by volume), within a supercritical gas saturation process. This method enhances fragrance solutions in hours to days (e.g., 1 hour to 168 hours at 50-300 bar, 31° C. to 60° C.), leveraging the tunable properties of supercritical gas (e.g., CO2) to improve solubility, potency, and longevity across all fragrance ingredients, unlike traditional maturation's reliance on prolonged solvent integration. This rapid enhancement establishes a transformative approach to fragrance production.

This transformative approach employs supercritical gas saturation within a pressure reactor, reducing the maturation timeline from weeks or months to hours or days (1 hour to 168 hours at 50-300 bar and 31° C. to 60° C.). By harnessing the high diffusivity of supercritical gas (e.g., CO2), the method enhances fragrance solutions rapidly and uniformly, bypassing the slow, passive diffusion of traditional maturation. This improves solubility, potency, and longevity of fragrance compositions, providing a scalable, controlled alternative that minimizes production delays and resource demands inherent in conventional perfumery.

The fragrance composition resulting from this supercritical gas saturation process may be formulated into diverse product categories, such as perfumes, eaux de toilette, bath preparations, environmental fragrances, or mixtures thereof, depending on application needs. For perfumery or related uses, the composition can adopt various physical forms, including liquid, gelled, viscous, solid, or combinations thereof, enabling versatile delivery tailored to consumer preferences while retaining the enhanced solubility, potency, and longevity achieved through this method, paving the way for broader adaptations in future formulations.

Central to this rapid process, liquid ethanol (10%-99% by volume) delivers an immediate fragrance release post-application, while its use in supercritical gas saturation enhances fragrance solutions for sustained solubility, potency, and longevity across all fragrance ingredients. Unlike traditional maturation's dependence on slow solvent diffusion over weeks or months, this method leverages ethanol's properties within a pressure reactor to achieve a vibrant, long-lasting fragrance composition in hours to days (1 hour to 168 hours), meeting consumer demands for efficiency and quality without heavy reliance on base notes.

Beyond perfumery, supercritical fluid technologies have advanced since the 1970s, driven by environmental awareness and sustainable industrial practices across food, pharmaceutical, and cosmetic sectors. Supercritical gases, such as carbon dioxide (CO2), are widely used in these fields for extraction processes—e.g., isolating lavender oil from flowers or caffeine from coffee beans—aiming to remove and purify specific compounds from a matrix, distinct from the saturation process herein, which enhances fragrance solutions. Unlike extraction or isolation, this invention employs supercritical gas saturation within a pressure reactor to improve the solubility, potency, and longevity of fragrance compositions. This method infuses liquid ethanol with supercritical gas (e.g., CO2) to enhance solubility—demonstrated by GC-MS increases of at least 4%-7% (e.g., Farnesol+7.83%)—reducing traditional maturation times from weeks or months to hours or days (1 hour to 168 hours). An unexpected benefit may also arise, as the supercritical gas could reduce volatile impurities in ethanol (e.g., fermentation residues), enhancing scent purity compared to conventional maturation. This dual action—solubility enhancement and potential residue reduction—distinguishes the invention from prior art, offering a transformative approach to fragrance production that prioritizes integration over isolation.

In contrast, the present invention avoids extraction or isolation, instead enhancing the solubility, potency, and longevity of fragrance compositions through a supercritical gas saturation process. This method leverages supercritical gas (e.g., CO2) and liquid ethanol within a pressure reactor to improve the solvent's ability to dissolve and integrate fragrance ingredients, yielding a more potent and long-lasting fragrance solution. It provides significant advantages in solubility, particularly for fragrance ingredients difficult to dissolve via traditional maturation.

Unlike traditional extraction methods where supercritical gases (e.g., CO2) isolate compounds from natural sources (e.g., lavender oil from flowers), this invention leverages supercritical gas saturation with liquid ethanol in a pressure reactor. This method enhances the solubility, potency, and longevity of initial fragrance solutions by increasing the uptake and integration of fragrance ingredient components into the ethanol and may further incorporate raw fragrance plant materials within the same supercritical environment. By saturating the solution, it ensures optimal solubility, potency, and longevity of the resulting fragrance composition.

Saturation in this context contrasts with extraction. While extraction removes compounds from a material using supercritical gases (e.g., CO2), this supercritical gas saturation process infuses liquid ethanol with supercritical gas (e.g., CO2) in a pressure reactor to enhance its ability to dissolve and integrate fragrance ingredients. Rather than isolating compounds, it amplifies the solubility, potency, and longevity of initial fragrance solutions, yielding a more potent and long-lasting fragrance composition.

Supercritical fluid technology offers new possibilities for enhancing the solubility, potency, and longevity of fragrance compositions. Supercritical gases, such as CO2 or N2, become particularly effective when used with liquid ethanol in a pressure reactor. This combination enhances solvent density, polarity, and interactions like hydrogen bonding, crucial for dissolving and integrating fragrance ingredients. The invention leverages these properties through a supercritical gas saturation process, operating at 50 bar to 300 bar and 31° C. to 60° C., where CO2 reaches its supercritical state at 74 bar and 31° C., while ethanol remains liquid to preserve fragrance integrity. This range optimizes solubility and enhancement of fragrance solutions during processing.

Supercritical gases, such as CO2 with a critical point at 31.1° C. and 74 bar, exhibit a unique combination of liquid-like densities (e.g., 0.6-1.0 g/cm³) and gas-like diffusivities (e.g., 10-3 cm²/s). These properties enable enhanced penetration and dissolution compared to traditional solvents, making them effective media for the supercritical gas saturation process. The low critical temperature of CO2, along with its non-toxicity, reduced environmental impact compared to traditional organic solvents, and non-flammability, positions supercritical gases as sustainable options, a versatility amplified by liquid ethanol's role in enhancing fragrance solutions within this method.

The solubility enhancement of this process leverages the pressure-dependent density of supercritical gases (e.g., CO2), a principle observed in applications like pharmaceutical dispersions where increased pressure (e.g., 250 bar) boosts solvent density and dissolution. Operating at 50 bar to 300 bar, with an example at 105 bar, this supercritical gas saturation method maximizes the solubility of fragrance ingredients in liquid ethanol, where higher gas density correlates with enhanced integration, improving potency and longevity of enhanced fragrance solutions without traditional maturation constraints.

Liquid ethanol (10%-99% by volume) serves as an effective solvent, enhancing the solubility, potency, and longevity of fragrance ingredients within initial fragrance solutions. When used with supercritical gas (e.g., CO2) in a pressure reactor, it enables the solution to dissolve and integrate a higher concentration of fragrance ingredients. The supercritical gas saturation process amplifies the solubility and interaction between ethanol and fragrance ingredients, and upon application, ethanol's rapid evaporation leaves a greater number of fragrance molecules on the skin, intensifying the potency and longevity of the fragrance composition. This method leverages supercritical gas properties to optimize solubility and release.

Supercritical gases like CO2, widely used due to its chemical stability, inertness, and high-purity availability at low cost, offer adjustable physical properties such as density, viscosity, and diffusivity through temperature and pressure modifications. For CO2, operating at 50 bar to 300 bar and 31° C. to 60° C.—near its critical point of 74 bar and 31.1° C.—enables significant variations in density and solubility. These characteristics, blending low viscosity and high diffusivity similar to gases with high density and solvent power akin to liquids, make supercritical gases (e.g., CO2 or N2) versatile for the supercritical gas saturation process. CO2's safety, as recognized by regulatory standards, further enhances its appeal. This invention leverages these properties to enhance the solubility, potency, and longevity of enhanced fragrance solutions.

When employing supercritical gases such as CO2, N2, or their mixtures with liquid ethanol (10%-99% by volume) in a pressure reactor, the combination enhances the solubility of fragrance ingredients within initial fragrance solutions. The supercritical gas increases the solvent's density, polarity, and interactions like hydrogen bonding, optimizing dissolution and integration. Ethanol's safety and compatibility with supercritical gases (e.g., CO2) further support this supercritical gas saturation process.

Based on the foregoing, improved methods for enhancing solubility, potency, and longevity of a fragrance composition are greatly needed.

### SUMMARY OF THE INVENTION

Unlike extraction methods that isolate compounds, this invention enhances the solubility, potency, and longevity of fragrance compositions by saturating the initial fragrance solution with supercritical gas in a pressure reactor, amplifying the integration of fragrance ingredients in situ. As a non-limiting example, the initial fragrance solution is prepared comprising liquid ethanol (10%-99% by volume) and at least one fragrance ingredient (e.g., single compounds, natural or synthetic mixtures, or blends), optionally with co-solvents such as propylene glycol (0.1% to 10% by volume). After purging the pressure reactor with a gas capable of achieving a supercritical state (e.g., CO2 or N2) to remove air, this solution is saturated at 50 bar to 300 bar and 31° C. to 60° C. (e.g., 105 bar and 33° C.) via incremental pressurization (e.g., 5-10 bar steps, 5-20 L/min flow rate), optionally incorporating raw fragrance plant materials. Beyond traditional perfumery types such as parfum, eau de parfum, eau de toilette, eau de cologne, and eau fraîche, this method may apply to products like body sprays, deodorants, wet towels, cosmetic compositions, household cleaners, and air fresheners. Optional additives, such as antioxidants, chelating agents, UV filters, preservatives, thickeners, fragrance microcapsules, moisturizers, humectants, emollients, opacifiers, pigments, colorants, dyes, or antifoams, may enhance stability, appearance, or functionality of the fragrance composition.

Gas Chromatography-Mass Spectrometry (GC-MS) testing, per claim 8, validated improvements over a fragrance solution control sample processed by traditional maturation, focusing on example fragrance ingredients—Farnesol, Linalool, Citronellol, and Limonene—selected for their distinct polar and non-polar profiles and clear identifiability in GC-MS analysis, unlike complex natural mixtures (e.g., rose oil) with multiple peaks. Results showed: Farnesol with a total area of 926,234,591.28 counts·min (+7.83% vs. control) and standard deviation of 0.2251 (−20.50%), indicating consistency; Linalool at 235,785,602.73 counts·min (+7.10%) with a standard deviation of 0.2785 (+64.72%), reflecting dynamic retention; Citronellol at 253,963,531.01 counts·min (+4.98%) with a standard deviation of 0.2222 (+36.81%); and Limonene at 222,954,354.76 counts·min (+4.54%) with a standard deviation of 0.3486 (+13.40%). These findings confirm enhanced solubility, potency, and longevity of the enhanced fragrance solution, supporting applications in perfumery and related products.

Distinct from prior art extraction methods using supercritical gases (e.g., CO2) to isolate compounds from natural sources, this supercritical gas saturation process integrates fragrance ingredients into liquid ethanol, achieving solubility increases of at least 4%-7% (e.g., Farnesol+7.83% per GC-MS) at an example pressure of 105 bar, reducing traditional maturation times from weeks or months to hours or days (1 hour to 168 hours). Operating within 50 bar to 300 bar, the pressure reactor sustains effective saturation, with higher pressures (e.g., 200 bar) potentially boosting solubility to 8%-15% or more, driven by increased gas density and enhanced interactions, as shown in preliminary trials. An unexpected benefit may emerge: supercritical gas (e.g., CO2) could reduce volatile ethanol impurities during saturation, enhancing scent purity beyond traditional maturation. This combination of rapid integration, amplified solubility, and potential impurity reduction highlights the invention's novelty and non-obviousness over conventional maturation and extraction techniques.

The supercritical CO2 saturation process offers compelling industrial applicability by introducing a high-pressure enhancement technique to fragrance manufacturing, an industry traditionally reliant on ambient-condition processes. Unlike conventional perfume production, which uses prolonged maturation in standard mixing vessels, this method leverages pressure reactors—equipment more typical in pharmaceutical sterilization—with minimal adaptation, such as adding CO2 injection systems and pressure controls (e.g., settings at 105 bar or 205 bar to maintain targets of 100 bar or 200 bar). By slashing maturation times from weeks or months to hours or days (optimally 24-48 hours), it offers manufacturers a scalable solution that could reduce production costs through reduced production timelines, thereby freeing up working capital, as well as reducing overall storage needs. The process boosts fragrance potency and solubility (e.g., 4-7% at 100 bar target, 8-15% or more at 200 bar target per GC-MS), appealing to both artisanal perfumers seeking premium scents and industrial producers aiming for efficiency. Beyond perfumery, the method could enhance aromatherapy oils by increasing retention of a wide range of fragrance compounds—spanning polar and non-polar profiles—for amplified therapeutic effects or improve scented candles by optimizing the release and burn consistency of complex, multi-ingredient fragrance blends. While pressure reactors are not yet commonplace in fragrance manufacturing, their adoption through this invention provides a practical, cost-effective pathway to revolutionize scented product development across diverse applications.

The supercritical gas saturation process offers compelling industrial applicability by introducing a high-pressure enhancement technique to fragrance production, traditionally reliant on ambient-condition maturation. Unlike conventional methods using prolonged maturation in standard vessels, this method leverages pressure reactors—equipment more typical in pharmaceutical sterilization—adaptable with gas injection and pressure controls (e.g., 105 bar) to operate at 50 bar to 300 bar and 31° C. to 60° C. Reducing maturation times from weeks or months to hours or days (1 hour to 168 hours), it provides a scalable solution that may lower production costs, timelines, and storage needs. It enhances solubility, potency, and longevity of enhanced fragrance solutions (e.g., 4%-7% solubility increase at 105 bar per GC-MS, potentially 8%-15% or more at 200 bar), appealing to artisanal perfumers seeking premium fragrance compositions and industrial producers targeting efficiency. Beyond perfumery, it may improve aromatherapy oils by enhancing retention of diverse fragrance ingredients spanning polar and non-polar profiles or optimize scented candles by improving release consistency of complex blends. While pressure reactors are uncommon in fragrance production, their adoption through this invention offers a practical, cost-effective pathway to advance scented product development across applications.

Upon gradual depressurization in controlled increments within the pressure reactor, the supercritical gas reverts to a gaseous state, leaving fragrance ingredients integrated within the liquid ethanol of the enhanced fragrance solution. This supercritical gas saturation process enhances the solubility, potency, and longevity of the fragrance composition, enabling greater uptake and integration of fragrance ingredients compared to a fragrance solution control sample processed by traditional maturation, with an improved release profile.

In an alternative approach to enhancing the solubility, potency, and longevity of initial fragrance solutions, the pressure reactor may include a designated saturation chamber. In this method, liquid ethanol (10%-99% by volume), optionally with co-solvents such as propylene glycol (0.1% to 10% by volume) and at least one fragrance ingredient, is introduced into the chamber before the supercritical gas saturation stage. The chamber is then sealed, and the pressure reactor is adjusted to achieve supercritical conditions (e.g., 50 bar to 300 bar and 31° C. to 60° C.) for a gas such as CO2 or N2.

During this stage, a supercritical gas (e.g., CO2 or N2) is introduced into the saturation chamber, where it saturates the liquid ethanol and fragrance solution. This supercritical gas saturation process enhances the ethanol's solvent properties, increasing its ability to dissolve and integrate fragrance ingredients within the fragrance solution.

Following saturation, additional fragrance ingredients may be introduced into the chamber, dissolving into the supercritical gas-saturated liquid ethanol within the fragrance solution, which exhibits enhanced solvating power under supercritical conditions (e.g., 50 bar to 300 bar and 31° C. to 60° C.). This supercritical gas saturation process ensures a uniform and potent fragrance solution.

The resulting fragrance solution, with supercritical gas-saturated liquid ethanol and integrated fragrance ingredients, forms a uniform mixture. This alternative supercritical gas saturation method enhances the solubility, potency, and longevity of the fragrance composition, offering flexibility in production and compatibility with other approaches described herein.

Another optional method for enhancing the potency and solubility of the final fragrance product involves dosing the pre-mixed perfume, which consists of liquid ethanol and fragrance additives, directly into the pressure reactor. In this method, the pre-mixed perfume is introduced into a high-pressure-resistant container within the pressure reactor. And still in another optional method, as was described previously, the pressure reactor can be equipped with a designated saturation chamber for receiving the pre-mixed perfume.

Another alternative method for enhancing the solubility, potency, and longevity of the fragrance composition involves introducing a pre-mixed fragrance solution, comprising liquid ethanol (10%-99% by volume) and at least one fragrance ingredient, directly into the pressure reactor. In this approach, the pre-mixed solution is placed into a high-pressure-resistant container within the reactor, or, as an additional variation described previously, into a designated saturation chamber for supercritical gas saturation.

The pressure reactor is then prepared for the supercritical gas saturation stage, adjusting temperature and pressure to achieve supercritical conditions (e.g., 50 bar to 300 bar and 31° C. to 60° C.) for a gas such as CO2 or N2. Once these conditions are reached, the supercritical gas is introduced into the reactor to saturate the pre-mixed fragrance solution.

The supercritical gas enhances the solvency of the liquid ethanol within the pre-mixed fragrance solution, enabling it to dissolve and integrate a higher concentration of fragrance ingredients. This supercritical gas saturation process improves the solubility, potency, and longevity of the fragrance composition, surpassing traditional maturation by enhancing uptake and integration of fragrance ingredients, resulting in a more intense and long-lasting product.

This alternative method enhances the performance of initial fragrance solutions by leveraging the properties of supercritical gas (e.g., CO2 or N2), reducing traditional maturation times from weeks or months to hours or days (1 hour to 168 hours). It serves as a flexible step in fragrance production, enabling manufacturers to improve the solubility, potency, and longevity of the fragrance composition efficiently.

In an alternative embodiment, the supercritical gas saturation process may directly process raw fragrance plant materials (e.g., jasmine, rose, lavender, vetiver) typically used in perfumery, optionally reducing or bypassing traditional maceration. These materials are placed with the fragrance solution, comprising liquid ethanol (10%-99% by volume) and optionally co-solvents like propylene glycol (0.1% to 10% by volume), in a stainless-steel basket or perforated container within the pressure reactor. As a non-limiting example, the reactor is saturated with supercritical gas (e.g., CO2 or N2) at 50 bar to 300 bar and 31° C. to 60° C. (e.g., 105 bar and 33° C.) for 1 hour to 168 hours, using incremental pressurization (e.g., 5-10 bar steps, 5-20 L/min flow rate). During saturation, the supercritical gas integrates aromatic compounds from the plant materials into the fragrance solution, leveraging high diffusivity and solvent power to enhance the fragrance composition in a single step. Depending on the materials and desired profile, this method may streamline production, yielding a potent and uniform enhanced fragrance solution surpassing traditional maturation. Post-saturation, the solution undergoes controlled depressurization, filtration, and homogenization, producing a tailored fragrance composition.

Other features and advantages of the present invention will immediately be recognized by persons of ordinary skill in the art with reference to the accompanying drawings and detailed description of exemplary embodiments as discussed herein.

### BRIEF DESCRIPTION OF THE DRAWINGS

The accompanying drawings illustrate various aspects and embodiments of the present invention disclosed herein but should not be construed as restricting the scope of the invention in any manner. In the drawings, like reference numerals refer to the same or similar elements or components.

FIG. 1 is a logical flow diagram illustrating the steps of a method for enhancing solubility, potency, and longevity of a fragrance composition, according to the present invention.

FIG. 2 illustrates a schematic overview of a pressure reactor for producing an enhanced fragrance solution comprising enhanced solubility, potency, and longevity.

### DETAILED DESCRIPTION OF THE INVENTION

Various aspects and embodiments of the methods of the present invention are now described in detail. Reference is made to the drawings, wherein like numerals refer to the same or similar parts throughout. The present invention is intended for enhancing solubility, potency, and longevity of a fragrance composition. The following detailed description elaborates on aspects of the supercritical gas saturation process for enhancing fragrance compositions, as set forth in the claims. Although the methods of the present invention are described primarily with respect to fragrance compositions, the present invention may be readily adapted other applications as well without departing from the scope of this invention. Hence, a myriad of other applications of the present invention are contemplated.

Unless explicitly stated otherwise, one of ordinary skill in the art would understand that features, structures, or characteristics of any embodiment described herein may be combined with those of other embodiments to enhance the solubility, potency, and longevity of fragrance solutions. As used herein, ‘initial fragrance solution’ refers to the pre-process mixture of liquid ethanol in an amount of 10% to 99% by volume, at least one fragrance ingredient, and optionally at least one co-solvent, prepared for supercritical gas saturation. ‘Enhanced fragrance solution’ refers to the fragrance solution recovered after supercritical gas saturation, exhibiting enhanced solubility, increased potency, and greater longevity. ‘Fragrance solution control sample’ refers to a fragrance solution processed by traditional maturation methods, serving as a benchmark for comparison.

The steps of the supercritical gas saturation process may be presented as discrete actions in a sequence designed to clarify the invention, as claimed. However, the order of the method steps in the description does not imply that these steps are strictly order-dependent. Specifically, the steps may be performed in a different sequence than presented, unless otherwise specified. Additional steps may be included, or described steps omitted, in alternative embodiments enhancing the solubility, potency, and longevity of fragrance compositions.

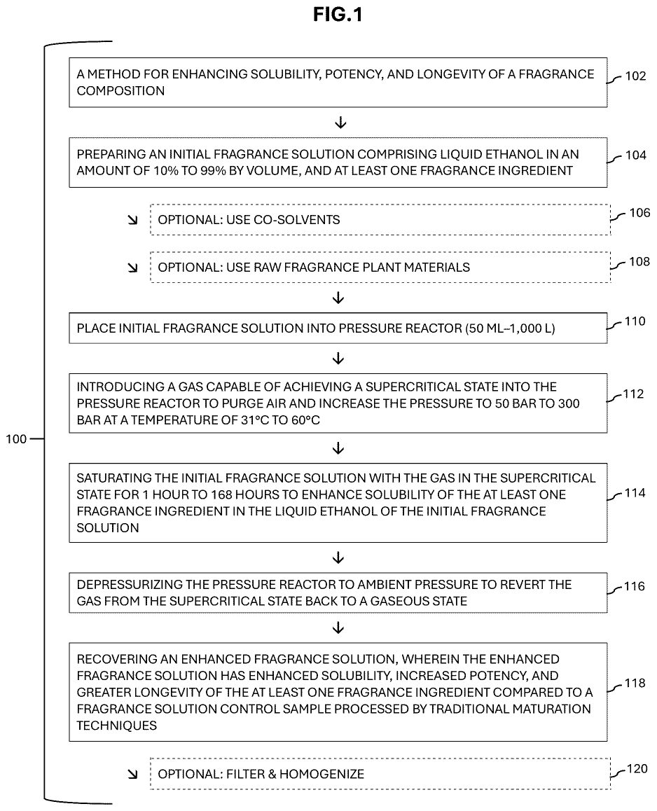

The following exemplary methods describe processes for enhancing solubility, potency, and longevity of a fragrance composition using a supercritical gas saturation process, as illustrated in the accompanying figures. FIG. 1 depicts a logical flow diagram of one such method, detailing the steps of preparing an initial fragrance solution, processing it in a pressure reactor with supercritical gas, and recovering an enhanced fragrance solution with improved properties compared to traditional maturation techniques. These methods leverage controlled pressure and temperature conditions to achieve rapid and efficient fragrance enhancement, as further elaborated in the descriptions below.

FIG. 1 depicts a logical flow diagram illustrating an exemplary method, denoted as step 100, for enhancing solubility, potency, and longevity of a fragrance composition in accordance with an embodiment of the present invention, comprising steps 102 through 120. Step 102 represents the overarching method for enhancing solubility, potency, and longevity of a fragrance composition, encapsulating the sequence of actions detailed in subsequent steps. Step 104 depicts preparing an initial fragrance solution comprising liquid ethanol in an amount of 10% to 99% by volume, and at least one fragrance ingredient, such as single compounds (e.g., Linalool), nature identical compounds, natural or synthetic mixtures (e.g., lavender oil), or blends. Step 106 depicts the optional use of co-solvents, such as but not limited to propylene glycol (0.1% to 10% by volume), to further increase the solvating properties being applied to the initial fragrance solution. Step 108 depicts the optional incorporation of raw fragrance plant materials (e.g., jasmine, rose) into the pressure reactor in substitution of the at least one fragrance ingredient to enhance the raw fragrance plant materials' aromatic profile. Step 110 depicts placing the initial fragrance solution into a pressure reactor with a capacity of 50 mL to 1,000 L, designed to withstand high-pressure conditions. Step 112 depicts introducing a gas capable of achieving a supercritical state, such as carbon dioxide (CO2) or nitrogen (N2), into the pressure reactor to purge air and increase the pressure to 50 bar to 300 bar at a temperature of 31° C. to 60° C., establishing supercritical conditions. Step 114 depicts saturating the initial fragrance solution with the gas in the supercritical state for 1 hour to 168 hours to enhance solubility of the at least one fragrance ingredient, or the substituted raw fragrance plant materials, in the liquid ethanol of the initial fragrance solution, leveraging the gas's high diffusivity and solvent power. Step 116 depicts depressurizing the pressure reactor to ambient pressure in controlled increments (e.g., 5-10 bar steps over 3-4 hours) to revert the gas from the supercritical state back to a gaseous state, preserving the solution's integrity. Step 118 depicts recovering an enhanced fragrance solution, wherein the enhanced fragrance solution exhibits enhanced solubility, increased potency, and greater longevity of the at least one fragrance ingredient, or the substituted raw fragrance plant materials, compared to a fragrance solution control sample processed by traditional maturation techniques, as evidenced by GC-MS analysis (e.g., Farnesol+7.83% total area). Step 120 depicts the optional post-processing of filtering and homogenizing the enhanced fragrance solution to ensure clarity and uniformity for applications such as perfumes or air fresheners.

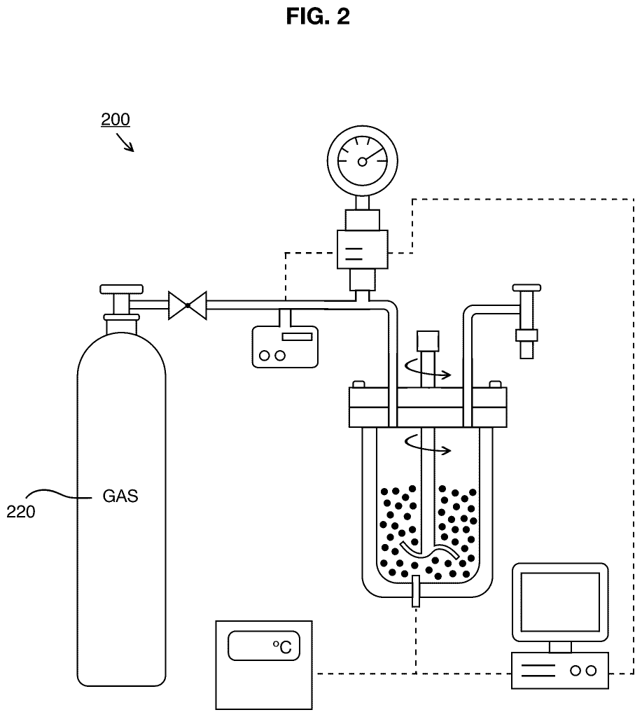

FIG. 2 illustrates a schematic overview of a pressure reactor system, denoted as 200, for producing an enhanced fragrance solution with enhanced solubility, potency, and longevity in accordance with an embodiment of the present invention. The system includes one or more gas tanks 220 containing a gas, such as CO2, N2, CO2+N2 mixtures, or other suitable gases, which is introduced into the reactor via a pressure valve and related components to purge air and achieve a supercritical state at pressures of 50-300 bar and temperatures of 31° C.-60° C., integrating fragrance ingredients and optional co-solvents into an initial fragrance solution to produce the enhanced fragrance solution.

The first step in the supercritical gas saturation process prepares the initial fragrance solution to enhance its solubility, potency, and longevity, as described in claim 1 and below. For example, the initial fragrance solution comprises liquid ethanol (10%-99% by volume), typically fragrance-grade ethyl alcohol (e.g., 200 proof) denatured with agents like tertiary butanol or denatonium benzoate, and at least one fragrance ingredient-such as single compounds (e.g., Linalool), natural or synthetic mixtures (e.g., lavender oil), or blends (e.g., floral accords)—with optional co-solvents like propylene glycol (0.1% to 10% by volume). Fragrance-grade ethanol, valued for its solvent properties and rapid evaporation upon application (e.g., 10-30 seconds at 25° C.), leaves fragrance ingredients on the skin, while co-solvents like propylene glycol may enhance viscosity (e.g., 41 cP vs. ethanol's 1.1 cP) or stability. Together, these high-purity components increase ethanol's solvating capacity in the pressure reactor, improving uptake of diverse fragrance ingredients beyond traditional maturation limits. The initial fragrance solution is then placed into the pressure reactor, where a supercritical gas (e.g., CO2 or N2) is introduced to purge air and increase the pressure to 50 bar to 300 bar at 31° C. to 60° C. (e.g., 105 bar and 33° C.). The solution is saturated with the supercritical gas for 1 hour to 168 hours, followed by gradual depressurization in controlled increments (e.g., over 1-4 hours) to revert the gas to a gaseous state, recovering the enhanced fragrance solution with preserved solubility, potency, and longevity.

In exemplary embodiments for bench-scale use, a pressure reactor like the Parr Series 4520 Bench Top Reactor (e.g., 1- or 2-liter capacity) may be utilized, which is constructed from Stainless Steel and rated for pressures up to 1900 psig (131 bar) at temperatures such as 33° C., within the 50 bar to 300 bar and 31° C. to 60° C. range of claim 1. The pressure reactor includes fittings like a gas inlet valve with dip tube, a liquid sampling valve, and a safety rupture disc, enabling controlled introduction and saturation of supercritical gas (e.g., CO2 or N2). The design, with options for fixed or removable heads and seals (e.g., PTFE gaskets or FKM O-rings rated to 225° C.), supports the initial fragrance solution under these conditions. Stirring via a variable speed motor (e.g., 0-600 rpm, up to 1700 rpm optional) ensures uniform mixing during saturation, while cooling coils may maintain temperature stability. At larger scales, such as commercial production, a reactor like the 500-liter Büchi Pilotclave, with enhanced capacity and agitation (e.g., turbine stirrer, 0-800 rpm or more), sustains efficacy across the 50 mL to 1,000 L range specified in claim 10, accommodating the process's scalability.

Molecular studies of supercritical CO2-ethanol systems reveal stable hydrogen-bonded aggregates where negative binding energies indicate robust interactions. In the supercritical gas saturation process at conditions like 50 bar to 300 bar and 31° C. to 60° C. (e.g., 105 bar and 33° C.), these interactions between liquid ethanol and fragrance ingredients within the initial fragrance solution enhance solubility and retention, as evidenced by GC-MS results (e.g., Farnesol+7.83% per claim 8), supporting efficacy across polar and non-polar fragrance profiles.

When liquid ethanol (10%-99% by volume) combines with a supercritical gas (e.g., CO2 or N2) in the pressure reactor, it enhances the solubility of fragrance ingredients within the initial fragrance solution, as per claim 1. This synergy improves integration, yielding a potent and long-lasting enhanced fragrance solution. The supercritical gas's properties, such as high diffusivity and efficient mass transfer, combined with ethanol, optimize the saturation process's efficacy. Adjusting temperature and pressure (e.g., 50 bar to 300 bar and 31° C. to 60° C.) tunes the solvent's diffusion coefficients, further enhancing solubility and retention of fragrance ingredients. This combination of ethanol and supercritical gas is key to achieving a robust enhanced fragrance solution.

Building on the supercritical gas saturation process, supercritical gas (e.g., CO2 or N2) may stabilize fragrance ingredients from the initial fragrance solution into amorphous powders, enhancing solubility for high-potency fragrance solids suited to perfumery applications under conditions like 50 bar to 300 bar and 31° C. to 60° C. for 1 hour to 168 hours. This optional adaptation could maintain fragrance ingredients in an amorphous state to boost solubility, producing potent fragrance powders derived from the enhanced fragrance solution, broadening the method's utility in fragrance compositions.

Scientific studies indicate that supercritical gas mixtures, such as CO2 with liquid ethanol (10%-99% by volume), exhibit unique solubility and dynamic properties that enhance solvent system performance within the initial fragrance solution. Similar effects may apply with N2 or CO2-N2 mixtures and optional co-solvents like propylene glycol (0.1% to 10% by volume), as used in claim 1. This enhancement is particularly effective for dissolving and integrating polar fragrance ingredients in the supercritical gas saturation process.

A key factor enhancing the solubility of polar fragrance ingredients in supercritical gas-ethanol mixtures (e.g., CO2 with liquid ethanol, 10%-99% by volume) within the initial fragrance solution is local composition enhancement (LCE), as utilized in claim 1. LCE describes the elevated concentration of ethanol molecules around polar fragrance ingredients in the supercritical gas saturation process, driven by hydrogen bonding and solvent aggregate formation. Molecular dynamics simulations reveal that ethanol creates robust hydrogen-bonded networks in the presence of supercritical gas, increasing local ethanol concentrations near fragrance ingredients. Similar effects may occur with optional co-solvents like propylene glycol (0.1% to 10% by volume) in CO2 or N2 environments. This aggregation fosters an environment that boosts dissolution of polar fragrance ingredients, overcoming solubility limitations of pure supercritical gas.

For fragrance ingredients with polar functional groups, this enhanced local environment within the initial fragrance solution, as described in claim 1, ensures optimal solubility during the supercritical gas saturation process. Hydrogen bonding between liquid ethanol (10%-99% by volume) or optional co-solvents like propylene glycol (0.1% to 10% by volume) and polar fragrance ingredients facilitates their effective integration. The aggregation of these solvent molecules in the supercritical gas (e.g., CO2 or N2) environment increases solvent density, further contributing to enhanced solubility.

The dynamic behavior of the CO2-ethanol mixture plays a significant role in the solvation process of the initial fragrance solution, with comparable effects anticipated for N2 or CO2-N2 mixtures with ethanol or other solvents. The research findings indicate that ethanol molecules exhibit slower redistribution dynamics compared to CO2 molecules, owing to the strong hydrogen bonding within ethanol aggregates. This results in a more stable local environment for polar solutes such as fragrance compounds. In a supercritical CO2 or N2-solvent mixture, the solvent molecules provide the necessary polarity and hydrogen-bonding capability to dissolve polar fragrance components, while CO2 or N2 contributes to the mixture's tunable solvating power through adjustments in pressure and temperature.

The ability of the solvent to form stable aggregates during the supercritical CO2 or N2 process enhances the retention and slow release of fragrance molecules within the initial fragrance solution. After the gas is removed, and upon application of the enhanced fragrance solution, the solvent (e.g., ethanol) evaporates from the skin, leaving behind a higher concentration of fragrance molecules. This results in a longer lasting and more intense olfactory experience, with solvents like propylene glycol potentially offering prolonged release due to higher viscosity. The strong intermolecular interactions between the solvent and fragrance molecules, which were established during the supercritical gas saturation process, help maintain the integration of the enhanced fragrance solution, even as the solvent evaporates.

The slower evaporation of the solvent in the enhanced fragrance solution can be attributed to several factors related to the supercritical CO2 or N2 environment during the supercritical gas saturation process. During production, the solvent forms stable aggregates within the initial fragrance solution in the presence of the supercritical gas, particularly through hydrogen bonding when using ethanol or propylene glycol. These aggregates cause the solvent to behave differently compared to its pure liquid state, altering its evaporation dynamics. Although the gas is no longer present in the enhanced fragrance solution, the solvent, when combined with fragrance molecules during the supercritical gas saturation process, must still overcome stronger intermolecular forces before evaporating, which slows down the evaporation rate. Additionally, the increased density of the initial fragrance solution, resulting from the supercritical gas saturation process, contributes to slower evaporation. A denser mixture evaporates more slowly because the molecules are more tightly packed, requiring more energy to transition into the gaseous phase. Furthermore, the hydrogen bonding interactions within the enhanced fragrance solution ‘hold back’ the solvent, reducing its evaporation rate. As the solvent evaporates from the enhanced fragrance solution, the cohesive interactions between fragrance molecules may further slowdown the process, effectively reducing the rate of the solvent's escape from the surface.

The properties of the supercritical CO2 or N2-solvent mixture within the initial fragrance solution can be fine-tuned by adjusting temperature and pressure, allowing manufacturers to control the solubility and release profile of the enhanced fragrance solution. By increasing the pressure or lowering the temperature, the density and solubility of the solvent in the initial fragrance solution can be enhanced, making it possible to dissolve higher concentrations of fragrance molecules without degradation. This tunable solvent system offers an advantage over traditional solvents, which lack the same flexibility and efficiency, improving the efficacy of the enhanced fragrance solution.

This method ensures that the enhanced fragrance solution is not only potent and long-lasting but also free from unwanted byproducts, owing to the stabilizing effects of the one or more liquid solvents.

Once the initial fragrance solution is prepared as described above, it is carefully transferred into a pressure reactor, such as one made of stainless steel, which is then securely sealed to prevent leakage or contamination during subsequent processing steps. The pressure reactor must be constructed from materials capable of withstanding extreme conditions of high pressure and temperature to process the initial fragrance solution effectively. Suitable materials include high-grade stainless steel, which offers excellent corrosion resistance and maintains integrity under supercritical conditions, or specialized glass or certain polymers designed for high-pressure environments.

Ensuring that the fragrance additives are evenly distributed within the initial fragrance solution under supercritical conditions can be challenging. To achieve uniform distribution, specialized systems such as stirring mechanisms or high-shear mixers, like an agitator with a magnetic coupling (e.g., capable of 5400 Nem torque and speeds up to 1200 rpm under 250 bar pressure), may be necessary within the pressure reactor. These systems ensure consistent mixing throughout the supercritical gas saturation process, thoroughly integrating the fragrance additives into the initial fragrance solution.

In order to preserve the integrity of the fragrance compounds within the initial fragrance solution while achieving optimal conditions for the supercritical gas, the supercritical gas saturation process is preferably carried out at temperatures between 31° C. and 60° C., and more preferably between 31° C. and 50° C. when using CO2, though adjustments may be made for N2 (critical point: −147° C., 34 bar) or CO2-N2 mixtures, depending on the specific gas, solvent (e.g., ethanol, propylene glycol), and fragrance components involved. This range ensures that volatile and sensitive fragrance components, particularly top notes, remain stable without degradation when using CO2 with ethanol in the initial fragrance solution. While 31° C. to 60° C. is provided as a non-limiting example for CO2, broader temperature ranges may apply to N2 or mixtures, with the key being to maintain a temperature above the critical point of the chosen gas while avoiding excessive heat that could compromise the integrity of the enhanced fragrance solution. In some cases, near-supercritical conditions, also known as sub-critical conditions, may be advantageous for saturating the initial fragrance solution with certain sensitive fragrance compounds, even if full supercritical conditions are not achieved. For example, temperatures can be maintained below the critical point of the gas, such as 15° C. to 30° C., while pressure can be adjusted as needed to achieve specific formulation goals for the enhanced fragrance solution.

The initial fragrance solution is placed into a pressure reactor-specialized equipment designed for supercritical fluid processes-either directly or via a container. After loading, the reactor is purged with CO2 (or potentially N2) to remove air, preventing oxidation of fragrance compounds, and then charged with supercritical CO2 or N2. The temperature is raised above the critical point (e.g., 31° C. for CO2,−147° C. for N2) and pressure increased beyond the critical threshold (e.g., 74 bar for CO2, 34 bar for N2), typically ranging from 50 to 300 bar as noted in the background, to achieve a supercritical state. In this state, the gas combines gas-like diffusivity with liquid-like solvating power, permeating the initial fragrance solution to enhance the solvent's properties and integrate fragrance compounds.

Saturation begins by introducing supercritical gas (e.g., CO2, N2, or mixtures) at 5-10 L/min into the initial fragrance solution, with pressure increasing in 5-10 bar steps from 1 bar to 50-200 bar (preferably 105 bar for CO2), stabilizing for 5-10 minutes per step at 31° C.-35° C. (optimally 33° C. for CO2). Flow rates adjust to 15-20 L/min until optimal solubility is achieved within the initial fragrance solution, confirmed by stable pressure (+0.1 bar) and a 5-10% density increase via a Coriolis flow meter, avoiding oversaturation or turbulence. Trials at 105 bar with CO2 and ethanol showed Farnesol and Linalool area increases of 7.83% and 7.10%, respectively, compared to fragrance solution control samples per claim 8, with similar enhancements expected for N2 or other solvents. This process ensures uniform dissolution of fragrance molecules, yielding a stable, homogeneous enhanced fragrance solution with optimized diffusion and potency. The high diffusivity of the liquid solvent in the supercritical gas significantly enhances mass transfer efficiency within the initial fragrance solution. This property is particularly beneficial for ensuring that fragrance molecules are uniformly dissolved and integrated into the initial fragrance solution, resulting in a stable and homogeneous enhanced fragrance solution. By carefully adjusting temperature and pressure conditions, the method optimizes diffusion coefficients, ensuring that the enhanced fragrance solution maintains its potency and consistency over time.

Post-saturation at 105 bar (for CO2, with adjustments for N2 or mixtures), depressurization reduces pressure in 5-10 bar increments over 3-4 hours (180-240 minutes when using CO2 with ethanol), with 5-10 minute stabilizations per step, maintaining 33° C. (+2° C. for CO2) to prevent rapid gas release or precipitation. Below 50 bar, temperature decreases to 25° C. at 1-2° C. per 5-10 minutes to minimize thermal shock (specific to CO2 with ethanol; temperature profiles may vary for N2 or solvents like propylene glycol). Cooling from gas expansion is offset by slight heating (e.g., jacketed reactor). After reaching 1 bar, the enhanced fragrance solution equilibrates for 10-15 minutes, then is filtered as needed, homogenized (1-15 minutes), and bottled in preferably light-resistant containers at 15° C.-25° C. Gas chromatography-mass spectrometry (GC-MS) testing of the enhanced fragrance solution prepared with supercritical CO2 and ethanol, conducted over five trials with adjustments for dilution to account for data variability, demonstrated Citronellol and Limonene area increases of 4.98% and 4.54%, and Farnesol's 20.50% standard deviation reduction, with comparable results anticipated for N2 or alternative solvents, including propylene glycol or blends such as ethanol and propylene glycol. Alternatively, GC-MS testing may employ a standard sweep across fragrance solution control samples and the enhanced fragrance solution, plotting approximately 60 to 80 peaks or more, with area differences calculated to confirm enhanced potency across a broader range of fragrance compounds, further validating the efficacy of the supercritical gas saturation process. This controlled depressurization is key to maintaining the solubility of the fragrance molecules within the enhanced fragrance solution, preventing phase separation or cloudiness in the enhanced fragrance solution.

The supercritical gas saturation process is preferably conducted using a high-pressure reactor system capable of withstanding 50-250 bar and operating at temperatures from −15° C. to 100° C. (to accommodate gases like N2), such as a pilot-scale pressure reactor with a 500-liter capacity. A suitable system includes a stainless-steel vessel (e.g., 850 mm I.D., 1420 mm height) with a welded heating jacket (−1/+4 bar, DN40 flange connections) for precise temperature control (e.g., 33° C.±2° C. for CO2, adjustable for N2), insulated with 100 mm mineral wool and stainless-steel cladding. The vessel features a bottom DN70 opening and a DN50 mushroom-shaped bottom valve for recovery of the enhanced fragrance solution. Agitation is provided by an in-line drive with a 4.0 kW AC motor (3-phase, 1400 rpm, Eex de II T4), coupled to a magnetic coupling (e.g., 5400 Nem torque, 250 bar, 200° C. max, ceramic ball bearings) and a turbine stirrer, adjustable from 50-800 rpm via a frequency converter. The cover plate includes multiple ports: DN25 and DN80 flanges with valves and blind flanges, a DN50 flange with an ATEX double Pt100 sensor, and a ½′ NPT pressure gauge (0-250 bar). A hydraulic lift with a stainless-steel frame and foot pump facilitates cover plate removal for maintenance. Accessories like a hollow stirrer shaft for gassing and maintenance tools enhance functionality. Materials contacting the initial fragrance solution (e.g., 316L/316Ti stainless steel, PTFE seals) ensure corrosion resistance and compatibility with various solvents (e.g., ethanol, propylene glycol, methanol) and supercritical gases (e.g., CO2, N2). Such equipment, exemplified by systems like the Buchiglas (Büchi AG) Pilotclave Type 3, supports the process's pressure, temperature, and mixing requirements, ensuring scalability and reproducibility across different gas and solvent combinations to produce the enhanced fragrance solution.

The aforementioned supercritical CO2 saturation process readily adapts to a range of pressure reactor sizes, accommodating production scales for the enhanced fragrance solution from laboratory experimentation to pilot testing and full-scale mass production. The process parameters—such as pressure (50-300 bar, optimally 105 bar), temperature (31° C.-60° C., optimally 33° C. for CO2), saturation duration (1-48 hours, optimally 24-48 hours), and depressurization rate (5-10 bar increments over 3-4 hours)—remain consistent across scales, with adjustments primarily in gas flow rates, agitation speeds, and reactor design to ensure uniform saturation of the initial fragrance solution and safety. For small-scale laboratory batches (e.g., 50 mL to 5 L), compact reactors with capacities of 100 mL to 10 L suffice. At the pilot scale, a 500 L reactor balances handling efficiency, energy consumption, and safety. This size supports efficient heat transfer and pressure control while minimizing operational costs and risks, making it ideal for commercial applications.

For larger-scale production of the enhanced fragrance solution, such as 1,000 L batches or greater, the supercritical gas saturation process scales proportionally with reactor modifications to accommodate increased volume and mass transfer demands. A 1,000 L high-pressure reactor may employ multiple impellers or magnetic couplings, and slightly extended stabilization periods per pressure step during pressurization and depressurization to prevent turbulence or incomplete saturation of the initial fragrance solution. Heat management remains critical at this scale, potentially requiring integrated cooling/heating jackets (−1/+4 bar capacity) and real-time monitoring (e.g., Coriolis flow meters for density increases of 5-10%) to ensure consistent solubility across the initial fragrance solution. For example, a 1,000 L batch of the initial fragrance solution saturated at 105 bar and 33° C. over 48 hours maintains the same potency gains (e.g., 4-7% per GC-MS per claim 8) as smaller scales, producing the enhanced fragrance solution, though energy consumption rises, offset by economies of scale in material throughput. Safety considerations, such as reinforced vessel walls (e.g., 316L stainless steel, 15-20 mm thickness) and automated pressure relief systems, are essential at this scale to mitigate risks of high-pressure operation. Across all scales—from lab (50 mL) to pilot (500 L) to mass production (1,000 L)—the supercritical gas saturation process retains its core advantage of reducing maturation times from weeks to hours or days, with the 500 L capacity offering an optimal compromise for most commercial applications of the enhanced fragrance solution. Smaller scales suit experimental development of the enhanced fragrance solution, while larger volumes support industrial demands (e.g., household cleaners, air fresheners), with reactor size and ancillary equipment (e.g., pumps, valves) tailored accordingly. Materials like 316L/316Ti stainless steel, PTFE seals, and corrosion-resistant alloys ensure compatibility with the initial fragrance solution and supercritical gases (CO2, N2) regardless of scale, maintaining the invention's efficacy and reproducibility in producing the enhanced fragrance solution.

To validate the efficacy and consistency of this scalable process, comparative GC-MS testing (five trials, adjusted for dilution to rule out variability) of the enhanced fragrance solution versus fragrance solution control samples assessed potency, retention, and stability using CO2 and ethanol. The study focused on four single-aroma-molecule fragrance compounds—Farnesol (musky, warm aroma), Linalool (floral, spicy scent), Citronellol (sweet, rosy fragrance), and Limonene (fresh, citrusy notes)—selected as examples for their distinct polar and non-polar profiles and ease of identification in GC-MS analysis. Complex natural ingredients (e.g., rose oil) were avoided due to their multiple peaks, which split data across varying polarities, complicating direct comparisons. This approach highlights the process's efficacy across both compound types:
Farnesol (Moderately Polar): Average 8.5626 (vs. 7.9632, +7.53%), total area 926,234,591.28 counts*min (vs. 858,937,743.39, +7.83%), height 20,900,326,003.63 counts (vs. 19,716,190,571.80, +6.00%), standard deviation 0.2251 (vs. 0.2832, −20.50%)—enhanced consistency.
Linalool (Polar): Average 6.0588 (vs. 5.686, +6.57%), total area 235,785,602.73 counts*min (vs. 220,165,988.59, +7.10%), height 5,527,973,539.71 counts (vs. 5,326,128,522.96, +3.79%), standard deviation 0.2785 (vs. 0.1691, +64.72%)—dynamic retention.
Citronellol (Polar): Average 6.0528 (vs. 5.7056, +6.08%), total area 253,963,531.01 counts*min (vs. 241,917,854.43, +4.98%), height 5,713,675,436.81 counts (vs. 5,577,678,389.91, +2.43%), standard deviation 0.2222 (vs. 0.1624, +36.81%)—complex retention.
Limonene (Non-Polar): Average 6.2012 (vs. 5.959, +4.06%), total area 222,954,354.76 counts*min (vs. 213,274,563.99, +4.54%), height 5,452,588,125.43 counts (vs. 5,304,484,455.59, +2.79%), standard deviation 0.3486 (vs. 0.3074, +13.40%)—moderate dynamics.

In an alternative approach, GC-MS testing may utilize a standard sweep across fragrance solution control samples and the enhanced fragrance solution, plotting approximately 60 to 80 peaks or more, with percent differences calculated between all peak areas of the fragrance solution control samples versus the enhanced fragrance solution when the delta is taken, further confirming the efficacy of the supercritical gas saturation process across a wide range of fragrance compounds. The process enhances potency, retention, and versatility of the enhanced fragrance solution, effectively handling both polar and non-polar compounds as demonstrated by these single-aroma-molecule examples with supercritical CO2 and ethanol. Similar enhancements are contemplated with N2, CO2+N2 mixtures, or other solvents like propylene glycol, methanol, or isopropanol, or blends thereof, including any possible combination with ethanol and propylene glycol, as used in claim 1.

Standard deviation differences between the enhanced fragrance solution and fragrance solution control samples provide valuable insights into the specialized supercritical CO2 saturation process's effects. For instance, Farnesol's 20.50% reduction in standard deviation (from 0.2832 to 0.2251) indicates improved batch-to-batch consistency, critical for commercial production where uniformity and predictability in outcomes of the enhanced fragrance solution are essential. This reflects precision and process uniformity, ensuring stability for non-polar compounds like Farnesol. In contrast, higher standard deviations for polar compounds like Linalool (+64.72%, from 0.1691 to 0.2785) and Citronellol (+36.81%, from 0.1624 to 0.2222) suggest enriched complexity, highlighting broader retention dynamics and diverse molecular interactions—desirable for creating rich, evolving profiles of the enhanced fragrance solution. Limonene, another non-polar compound, shows a moderate increase (+13.40%, from 0.3074 to 0.3486), balancing consistency with nuanced complexity. Increased standard deviation reflects diversity in retained isomers or volatiles, enhancing richness of the enhanced fragrance solution, while lower variability underscores precision and stability. This balance between consistency and complexity, tailored to each compound's chemical properties, underscores the process's versatility and context-sensitive approach in enhancing the enhanced fragrance solution.

The specialized supercritical CO2 saturation process demonstrates significant improvements in the enhanced fragrance solution across the tested compounds, with distinct impacts on retention and potency. Linalool exhibits the most significant improvement in total area (+7.10%) and average amount (+6.57%), with increased variability (+64.72%) suggesting the process retains a broader range of isomers or interactions, enriching the profile of the enhanced fragrance solution with complex and dynamic floral notes; higher height (+3.79%) indicates more pronounced peaks, enhancing the scent's presence. Citronellol shows a moderate improvement in concentration and retention (+6.08% average amount), with increased variability (+36.81%) reflecting dynamic retention of isomers or interactions, supported by higher total area (+4.98%) and height (+2.43%), which enrich the enhanced fragrance solution with complex floral notes and greater compound abundance. Farnesol displays notable improvement in retention and potency (+7.83% total area, +6.00% height), with decreased variability (−20.50%) demonstrating precise, uniform retention across trials, where the higher total area suggests consistent retention enhances potency without sacrificing complexity in the enhanced fragrance solution. Limonene's enhanced fragrance solution shows consistent potency and retention across all metrics (+4.54% total area, +2.79% height, +4.06% average), with a slight increase in variability (+13.40%) reflecting nuanced complexity while maintaining consistency, resulting in a richer, more robust profile of the enhanced fragrance solution. These outcomes highlight the process's ability to optimize both stability and richness, tailored to each compound's role in the enhanced fragrance solution.

The invention's versatility is evidenced by its ability to enhance the retention and potency of both polar and non-polar fragrance components within the initial fragrance solution, showcasing its capacity to manage a diverse range of chemical characteristics effectively. Polar compounds like Linalool and Citronellol, with hydroxyl groups that increase solubility in water-based systems but vulnerability to degradation, benefit from remarkable preservation, as seen in higher area counts, improved retention, and enhanced dynamics (e.g., +64.72% and +36.81% standard deviations), suggesting richer, more complex profiles of the enhanced fragrance solution. Non-polar compounds like Limonene and Farnesol, characterized by hydrocarbon chains or rings that heighten volatility, are effectively retained, with Farnesol showing improved consistency (−20.50% standard deviation) and Limonene balancing moderate dynamics (+13.40%) with robust outcomes (+4.54% total area). This dual efficacy-preserving polar ingredients' stability and complexity while ensuring non-polar ingredients' uniform retention-delivers a balanced, robust profile of the enhanced fragrance solution suitable for diverse applications, from perfumes and eaux de toilettes to household cleaners and air fresheners. By optimizing both polar (e.g., Citronellol) and non-polar (e.g., Limonene) compounds, the specialized supercritical CO2 saturation process outperforms conventional methods, offering unparalleled flexibility for commercial and industrial production of the enhanced fragrance solution.

This supercritical gas saturation process significantly increases the solubility of fragrance molecules within the initial fragrance solution, leading to improved potency and integration into the solvent. In instances when the fragrance concentrate and ethanol are not loaded directly into the pressure reaction chamber, a specialized container that enables the supercritical gas to enter is selected with specialized dosing ports or valves integrated into the container. These ports allow the gas (e.g., CO2, N2) to be introduced into the container from the pressure reactor's internal environment while maintaining the sealed state, thereby preventing any contamination or leakage. This ensures that the initial fragrance solution can be effectively saturated with the supercritical gas under controlled conditions. Alternatively, the container may be equipped with a selectively permeable membrane that allows the supercritical gas to diffuse into the container without compromising its sealed integrity. This method enables the gas to interact with the initial fragrance solution without directly opening the container. The pressure reactor may also feature a controlled injection system that introduces the supercritical gas directly into the sealed container. This system is designed to maintain the container's sealed environment while ensuring thorough saturation of the initial fragrance solution with the supercritical gas. In another alternative approach, the supercritical gas and fragrance compounds, including the solvent, are allowed to flow freely within a chamber of the pressure reactor, enabling direct interaction between them to produce the enhanced fragrance solution.

During the supercritical phase, additional fragrance additives can be introduced into the pressure reactor. This step is crucial for tailoring the profile of the enhanced fragrance solution to achieve the desired complexity and longevity. The pressure reactor is equipped with specialized dosing ports that allow for the controlled introduction of these additives without disrupting the supercritical environment. The additives are exposed to the supercritical gas-solvent mixture, where the enhanced solubility allows them to dissolve more efficiently than under normal conditions. The careful control of the additive integration process ensures that these additional components are evenly distributed throughout the initial fragrance solution, leading to a homogeneous mixture. This homogeneity is essential for the consistency of the enhanced fragrance solution, ensuring that each application delivers the same olfactory experience.

After the initial fragrance solution has been adequately saturated with the supercritical gas and the desired integration of additives has been achieved, the next step involves the controlled depressurization of the pressure reactor, as detailed above. This is a critical phase, as the transition from supercritical to ambient conditions must be carefully managed to avoid any destabilization of the initial fragrance solution. Once depressurization is complete, the enhanced fragrance solution is carefully recovered. At this stage, the enhanced fragrance solution, now improved in potency and longevity due to the supercritical gas saturation process, is ready for any desired post-processing steps.

Following the supercritical gas saturation and depressurization processes, the enhanced fragrance solution may undergo additional post-processing steps to ensure its stability and consistency. One common step is filtration, which helps remove any particulates or undissolved components that may have been introduced during the process. Filtration ensures that the enhanced fragrance solution is clear and free from any unwanted residues. Another crucial post-processing step is homogenization, which involves thoroughly mixing the enhanced fragrance solution to ensure that all components are uniformly distributed. This step is particularly important if the enhanced fragrance solution exhibits any signs of separation after depressurization. Homogenization can help to stabilize the enhanced fragrance solution, ensuring that the enhanced fragrance solution maintains a consistent scent profile and performance over time. These post-process handling steps are vital for ensuring the quality and reliability of the enhanced fragrance solution.

The method presented in this invention offers several key advantages over traditional fragrance formulation techniques. Firstly, the enhanced solubility achieved through the supercritical gas saturation process allows for a higher concentration of fragrance molecules in the initial fragrance solution's liquid solvent. This results in a more intense and potent enhanced fragrance solution that delivers a stronger olfactory impact. Additionally, the process may stabilize a greater number of fragrance molecules within the enhanced fragrance solution's ethanol-based solvent matrix, slowing their evaporation rate and resulting in a longer-lasting scent-potentially improved by co-solvents like propylene glycol, which increases overall solvating power and provides higher viscosity, as used in claim 3. The supercritical gas saturation process may also alter the release profile of the enhanced fragrance solution, bringing out certain notes more prominently and creating a more complex and layered scent experience. These benefits make the method particularly appealing for creating high-end, luxury enhanced fragrance solutions that stand out in the market.

Despite its benefits, the method presents several challenges. One primary challenge is maintaining consistency during the depressurization phase, where rapid pressure changes, if not carefully managed, could cause phase separation or precipitation of certain fragrance components, compromising the clarity and uniformity of the enhanced fragrance solution. Another challenge is ensuring material compatibility within the pressure reactor, as the initial fragrance solution must not react with the equipment to avoid contamination or degradation. This requires robust sealing and permeation design—for instance, the container must be sealed to retain the initial fragrance solution while allowing supercritical CO2 or N2 to permeate and saturate it. Alternatively, a dedicated chamber within the reactor could enable direct saturation of the initial fragrance solution with supercritical gas, bypassing the need for a semi-permeable container.

To address these challenges or tailor the process further, one alternative approach could involve processing the initial fragrance solution with an initial set of additives, depressurizing the system, and then reintroducing the enhanced fragrance solution with additional ingredients in subsequent stages, depending on the desired profile and stability requirements of the enhanced fragrance solution.

Thus, specific embodiments for enhancing solubility and potency of the enhanced fragrance solution using supercritical fluid saturation have been disclosed.

It should be apparent, however, to those skilled in the art that many more modifications besides those already described are possible without departing from the inventive concepts herein, including additional alternative structural and functional designs as disclosed from the principles herein. Thus, while embodiments and applications have been illustrated and described, it is to be understood that the disclosed embodiments are not limited to the precise construction and components disclosed herein. Various modifications, changes, and variations, which will be apparent to those skilled in the art, may be made in the arrangement, operation and details of the apparatus and methods disclosed herein without departing from the spirit and scope of the disclosure. Thus, it is intended that the present disclosure covers the modifications and variations of the embodiments disclosed above provided that the modifications and variations come within the scope of any claims and their equivalents.

The inventive subject matter, therefore, is not to be restricted except in the spirit of the appended claims. Moreover, in interpreting both the specification and the claims, all terms should be interpreted in the broadest possible manner consistent with the context. Throughout this specification, some embodiments have used the expressions “comprises,” “comprising,” “includes,” “including,” “has,” “having,” or any other variation thereof, all of which are intended to cover a non-exclusive inclusion. For example, a process, method, article, or apparatus that comprises a list of elements is not necessarily limited to only those elements but may include other elements not expressly listed or inherent to such process, method, article, or apparatus. In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise. Where the specification or claims refer to at least one of something selected from the group consisting of A, B, C, . . . and N, the text should be interpreted as requiring only one element from the group, not A plus N, or B plus N, etc.

As used herein any reference to any of “one embodiment” or “an embodiment,” “one variant” or “a variant,” and “one implementation” or “an implementation” means that a particular element, feature, structure, or characteristic described in connection with the embodiment, variant or implementation is included in at least one embodiment, variant or implementation. The appearances of such phrases in various places in the specification are not necessarily all referring to the same embodiment, variant or implementation.

It will be recognized that while certain aspects of the technology are described in terms of a specific sequence of steps of a method, these descriptions are only illustrative of the broader methods of the disclosure and may be modified as required by the particular application. Certain steps may be rendered unnecessary or optional under certain circumstances. Additionally, certain steps or functionality may be added to the disclosed implementations, or the order of performance of two or more steps permuted. All such variations are considered to be encompassed within the disclosure disclosed and claimed herein.

While the above detailed description has shown, described, and pointed out novel features of the disclosure as applied to various implementations, it will be understood that various omissions, substitutions, and changes in the form and details of the apparatus, article, method, or process illustrated may be made by those skilled in the art without departing from the disclosure. The foregoing description is of the best mode presently contemplated of carrying out the principles of the disclosure. This description is in no way meant to be limiting but rather should be taken as illustrative of the general principles of the technology. The scope of the disclosure should be determined with reference to the claims.

What is claimed is:

1. A method for enhancing solubility, potency, and longevity of a fragrance composition, comprising:
preparing an initial fragrance solution comprising liquid ethanol in an amount of 10% to 99% by volume, and at least one fragrance ingredient;
placing the initial fragrance solution into a pressure reactor;
introducing a gas capable of achieving a supercritical state into the pressure reactor to purge air and increase the pressure to 50 bar to 300 bar at a temperature of 31° C. to 60° C.;
saturating the initial fragrance solution with the gas in the supercritical state for 1 hour to 168 hours to enhance solubility of the at least one fragrance ingredient in the liquid ethanol of the initial fragrance solution;
depressurizing the pressure reactor to ambient pressure to revert the gas from the supercritical state back to a gaseous state; and
and recovering an enhanced fragrance solution, wherein the enhanced fragrance solution has enhanced solubility, increased potency, and greater longevity of the at least one fragrance ingredient compared to a fragrance solution control sample processed by traditional maturation techniques.

2. The method of claim 1, wherein the liquid ethanol is denatured alcohol present in an amount of 60% to 90% by volume.

3. The method of claim 1, wherein the initial fragrance solution further comprises at least one co-solvent, wherein the co-solvent is propylene glycol present in an amount of 0.1% to 10% by volume in the initial fragrance solution to enhance solvating capability of the liquid ethanol and to prevent precipitation of the at least one fragrance ingredient out of initial fragrance solution.

4. The method of claim 1, wherein the gas is carbon dioxide (CO2).

5. The method of claim 4, wherein the step of introducing the gas into the pressure reactor increases the pressure in the pressure reactor to a supercritical state of at least 74 bar up to 500 bar at a temperature of 31.1° C. to 60° C., and wherein the saturating step is performed for 1 hour to 168 hours, optionally with stirring of the fragrance solution.

6. The method of claim 1, wherein the gas is nitrogen (N2) or a mixture of carbon dioxide (CO2) and nitrogen (N2).

7. The method of claim 1, wherein the step of depressurizing the pressure reactor comprises gradually reducing the pressure in controlled increments to prevent destabilization of the enhanced fragrance solution.

8. The method of claim 1, wherein the enhanced solubility of the at least one fragrance ingredient in the enhanced fragrance solution is measurable by gas chromatography-mass spectrometry (GC-MS).

9. The method of claim 1, further comprises the step of placing raw fragrance plant materials into the pressure reactor with the initial fragrance solution, and wherein the gas in the supercritical state integrates one or more aromatic compounds from the raw fragrance plant materials into the initial fragrance solution during the saturating step to further enhance the fragrance composition.

10. The method of claim 1, wherein the pressure reactor is configured to process the fragrance solution at a volume of 50 mL to 1,000 L.

11. A method for enhancing solubility, potency, and longevity of a fragrance composition, comprising:
preparing an initial fragrance solution comprising liquid ethanol, at least one fragrance ingredient;
placing the initial fragrance solution into a pressure reactor;
introducing a gas capable of achieving a supercritical state into the pressure reactor to purge air and increase the pressure;
saturating the initial fragrance solution with the gas in the supercritical state to enhance solubility of the at least one fragrance ingredient in the liquid ethanol of the initial fragrance solution; and
depressurizing the pressure reactor to ambient pressure to revert the gas from the supercritical state back to a gaseous state; and
recovering an enhanced fragrance solution, wherein the enhanced fragrance solution has enhanced solubility, increased potency, and greater longevity of the at least one fragrance ingredient compared to a fragrance solution control sample processed by traditional maturation techniques.

12. The method of claim 11, wherein the liquid ethanol is denatured alcohol present in an amount of 10% to 99% by volume.

13. The method of claim 12, wherein the initial fragrance solution further comprises at least one co-solvent is propylene glycol present in an amount of 0.1% to 10% by volume in the initial fragrance solution to enhance solvating capability of the liquid ethanol and to prevent precipitation of the at least one fragrance ingredient out of the initial fragrance solution.

14. The method of claim 13, wherein the gas is carbon dioxide (CO2).

15. The method of claim 14, wherein the step of introducing the gas into the pressure reactor increases the pressure in the pressure reactor to a supercritical state of at least 50 bar up to 500 bar at a temperature of 31.1° C. to 60° C., and wherein the saturating step is performed for 1 hour to 168 hours, optionally with stirring of the fragrance solution.

16. The method of claim 15, wherein the gas is nitrogen (N2) or a mixture of carbon dioxide (CO2) and nitrogen (N2).

17. The method of claim 16, wherein the step of depressurizing the pressure reactor comprises gradually reducing the pressure in controlled increments to prevent destabilization of the enhanced fragrance solution.

18. The method of claim 17, wherein the enhanced solubility of the at least one fragrance ingredient in the enhanced fragrance solution is measurable by gas chromatography-mass spectrometry (GC-MS).

19. The method of claim 18, further comprises the step of placing raw fragrance plant materials into the pressure reactor with the initial fragrance solution, wherein the gas in the supercritical state integrates one or more aromatic compounds from the raw fragrance plant materials into the initial fragrance solution during the saturating step to further enhance the fragrance composition.

20. The method of claim 19, wherein the pressure reactor is configured to process the fragrance solution at a volume of 50 mL to 1,000 L.
